(19) United States
(12) Patent Application Publication
(10) Pub. No.: US 2011/0004057 A1
(43) Pub. Date: Jan. 6, 2011

(54) SYSTEMS AND METHODS FOR TRANSNASAL DILATION OF PASSAGEWAYS IN THE EAR, NOSE OR THROAT

(75) Inventors: Eric Goldfarb, Belmont, CA (US); John Y. Chang, Mountain View, CA (US); William M. Facteau, Mountain View, CA (US); Sivette Lam, Milpitas, CA (US); Hung V. Ha, San Jose, CA (US); Isaac J. Kim, San Jose, CA (US); Ketan P. Muni, San Jose, CA (US)

Correspondence Address: STEPTOE & JOHNSON - ACCLARENT, INC., 2121 AVENUE OF THE STARS, SUITE 2800, LOS ANGELES, CA, 90067, US

(73) Assignee: ACCLARENT, INC., Menlo Park, CA (US)

(21) Appl. No.: 12/793,352

(22) Filed: Jun. 3, 2010

Related U.S. Application Data
(63) Continuation-in-part of application No. 12/496,226, filed on Jul. 1, 2009.
Application No. 12/496,226 is a continuation-in-part of application No. 11/789,704, filed on Apr. 24, 2007.
Application No. 11/789,704 is a continuation-in-part of application No. 11/355,512, filed on Feb. 16, 2006.
Application No. 11/355,512 is a continuation-in-part of application No. 11/150,874, filed on Jun. 13, 2005, now abandoned.
Application No. 11/150,874 is a continuation-in-part of application No. 10/944,270, filed on Sep. 17, 2004, now abandoned.
Application No. 10/944,270 is a continuation-in-part of application No. 10/829,917, filed on Apr. 21, 2004, now Pat. No. 7,654,997.

(51) Int. Cl. A61M 29/02; A61B 1/00

(52) U.S. Cl. 600/106; 606/196

(57) ABSTRACT

A dilation catheter device and system for dilating an opening in a paranasal sinus and/or other passageways within the ear, nose or throat is disclosed. A dilation catheter device and system is constructed in a manner that facilitates ease of use by the operator and, in at least some cases, allows the dilation procedure to be performed by a single operator. Additionally, the dilation catheter device and system may be useable in conjunction with an endoscope and/or a fluoroscope to provide for easy manipulation and positioning of the devices and real time visualization of the entire procedure or selected portions thereof. In some embodiments, shaft markers are disposed on a shaft of the dilation catheter and have a light color to contrast with a dark color of the dilation catheter shaft. The high contrast between the markers and catheter shaft allows for easy viewing of the markers in low light and operation conditions.

### RELATED APPLICATIONS

This application is a continuation in part of U.S. patent application Ser. No. 11/789,704 entitled “Systems and Methods for Transnasal Dilation of Passageways in the Ear, Nose and Throat,” filed Apr. 24, 2007, which is a continuation in part of U.S. patent application Ser. No. 11/355,512 entitled “Devices, Systems and Methods Useable for Treating Frontal Sinusitis,” filed Feb. 16, 2006, which is a is a continuation in part of Ser. No. 11/150,874 entitled “Devices, Systems and Methods Useable for Treating Sinusitus,” filed on Jun. 10, 2005, which is a continuation in part of Ser. No. 10/944,270 entitled “Apparatus and Methods for Dilating and Modifying Ostia of Paranasal Sinuses and Other Intranasal or Paranasal Structures,” filed on Sep. 17, 2004, which is a continuation in part of Ser. No. 10/829,917 entitled “Devices, Systems and Methods for Diagnosing and Treating Sinusitis and Other Disorders of the Ears, Nose and/or Throat,” filed on Apr. 21, 2004, the entire disclosures of each such application being expressly incorporated herein by reference.

### FIELD OF INVENTION

The present invention relates generally to medical devices and methods and particularly to balloon catheters and other devices that may be inserted through the nose and used to dilate the ostia of paranasal sinuses for treatment of sinusitis.

### BACKGROUND

The paranasal sinuses are hollow cavities in the skull connected by small openings, known as ostia, to the nasal canal. Each ostium between a paranasal sinus and the nasal cavity is formed by bone covered by a layer of mucosal tissue. Normally, air passes into and out of the paranasal sinuses through the ostia. Also, mucus is continually formed by the mucosal lining of the sinuses and drains through the ostia and into the nasal canal.

Sinusitis is a general term that refers to inflammation in one or more of the paranasal sinuses. Acute sinusitis can be associated with upper respiratory infections or allergic conditions, which may cause tissue swelling and temporarily impede normal trans-ostial drainage and ventilation of the sinuses, thereby resulting in some collection of mucus and possibly infection within the sinus cavities. Chronic sinusitis is a long term condition characterized by persistent narrowing or blockage of one or more sinus ostia, resulting in chronic infection and inflammation of the sinuses. Chronic sinusitis is often associated with longstanding respiratory allergies, nasal polyps, hypertrophic nasal turbinates and/or deviated internasal septum. While acute sinusitis is typically caused by infection with a single pathogen (e.g., one type of bacteria, one type of virus, one type of fungus, etc.), chronic sinusitis is often associated with multiple pathogen infections (e.g., more than one type of bacteria or more than genus of microorganism).

Chronic sinusitis, if left untreated, can result in irreparable damage to the tissues and/or bony structures of the paranasal anatomy. The initial treatment of chronic sinusitis usually involves the use of drugs such as decongestants, steroid nasal sprays and antibiotics (if the infection is bacterial). In cases where drug treatment alone fails to provide permanent relief, surgical intervention may be indicated.

The most common surgical procedure for treating chronic sinusitis is functional endoscopic sinus surgery (FESS). FESS is commonly performed using an endoscope and various rigid instruments inserted through the patient's nostril. The endoscope is used to visualize the positioning and use of various rigid instruments used for removing tissue from the nasal cavity and sinus ostia in an attempt to improve sinus drainage.

Recently, a technique known as the Balloon Sinuplasty™ procedure and a system for performing the procedure have been developed by Acclarent, Inc. of Menlo Park, Calif. for treatment of sinusitis. A number of copending United States patent applications, including parent application Ser. No. 11/789,704, 11/355,512, 11/150,874, 10/944,270 and 10/829,917, describe various embodiments of the Balloon Sinuplasty™ procedure as well as various devices useable in the performance of such procedure. In the Balloon Sinuplasty™ procedure, a guide catheter is inserted into the nose and positioned within or adjacent to the ostium of the affected paranasal sinus. A guidewire is then advanced through the guide catheter and into affected paranasal sinus. Thereafter, a dilation catheter having an expandable dilator (e.g., an inflatable balloon) is advanced over the guidewire to a position where the dilator is positioned within the ostium of the affected paranasal sinus. The dilator is then expanded, causing dilation of the ostium and remodeling of bone adjacent to the ostium, without required incision of the mucosa or removal of any bone. The catheters and guidewire are then removed, and the dilated ostium allows for improved drainage from and ventilation of the affected paranasal sinus.

Parent application Ser. Nos. 11/789,704, 11/355,512, 11/150,874, 10/944,270 and 10/829,917 also describe methods for transnasal dilation of other passageways in the ear, nose and/or throat, such as the Eustachian tube and nasolacrimal duct.

It would be desirable to have improved methods, devices and systems for dilating paranasal sinus ostia. Ideally such improved methods, devices and systems would be easier to use and/or more effective than prior versions. At least some of these objectives will be met by the embodiments described below.

### SUMMARY

In one embodiment, there is provided a dilation catheter device and system that is useable for dilating the ostium of a paranasal sinus, or other passageway within the ear, nose or throat. This dilation catheter device and system is constructed in a manner that facilitates ease of use by the operator and, in at least some cases, allows the dilation procedure to be performed by a single operator, thereby minimizing the number of personnel required for the procedure. Additionally, the dilation catheter device and system of the present invention is useable in conjunction with an endoscope and/or a fluoroscope to provide for easy manipulation and positioning of the devices and real time visualization of the entire procedure or selected portions thereof. In some embodiments, an optional handle may be attached to the dilation catheter or to a guide catheter through which the dilation catheter is inserted and such handle may be graspable along with another device (e.g., an endoscope) by a single hand. In this manner, the operator may control the dilation catheter and another device (e.g., an endoscope) with one hand while being free to use his other hand for other purposes.

Further in one embodiment, there are provided systems for treating a disease or disorder of the ear, nose or throat of a human or animal subject. Such systems generally comprise a guide catheter and a working catheter. The working catheter is advanceable through the guide catheter. The guide catheter has a substantially rigid shaft and the working catheter has a proximal portion that is substantially rigid. The working catheter also has a distal portion that is more flexible than the substantially rigid proximal portion. The working catheter is sized relative to the guide catheter so that, at least when the distal portion of the working catheter is advanced out of a distal opening of the guide catheter and the working element is being used to perform a desired diagnostic or therapeutic task, only the substantially rigid proximal portion (or some portion thereof) will extend out of the proximal opening of the guide catheter. In some embodiments, the working catheter may additionally be sized relative to the guide catheter so that the working catheter is initially advanceable to a first position where its distal end of the working catheter has not yet emerged out of the distal end of the guide catheter but only the substantially rigid proximal portion of the working catheter is protruding out of the proximal end of the guide catheter.

Still further in accordance with another embodiment, there are provided sinus ostium dilation catheter devices that generally comprise an elongate catheter shaft having proximal shaft section that is substantially rigid and a distal shaft section that is more flexible than the proximal shaft section. In some embodiments, the proximal shaft section may extend along at least about 50% of the overall length of the device. A guidewire lumen extends through at least a portion of the catheter shaft to facilitate advancement of the catheter over a guidewire. A dilator is located on the distal shaft section, such dilator having a non-expanded configuration and an expanded configuration.

Still further in accordance with one embodiment, there are provided methods for dilating the ostia of paranasal sinus and other passageways within the ear, nose or throat of a human or animal subject. In general, such methods comprise the steps of a) inserting a guide catheter having a proximal end and a distal end through one of the subject's nostrils and positioning the guide catheter within or near the passageway to be dilated, b) inserting, through the guide catheter, a dilation catheter comprising i) an elongate catheter shaft having a proximal end, a distal end, a proximal shaft section that is substantially rigid and a distal shaft section that is more flexible than the proximal shaft section, ii) a guidewire lumen extending through at least a portion of the catheter shaft to facilitate advancement of the catheter over a that is substantially rigid and a distal shaft section that is more flexible than the proximal shaft section, ii) a guidewire lumen extending through at least a portion of the catheter shaft to facilitate advancement of the catheter over a guidewire and iii) a dilator located on the distal shaft section, said dilator being in a non-expanded configuration, c) positioning the dilator within the passageway and d) causing the dilator to expand to an expanded configuration, thereby dilating the passageway.

In still a further embodiment, a balloon dilation catheter device is provided that is useable for dilating an opening in a paranasal sinus. The dilation catheter device includes a catheter shaft having a longitudinal axis, an inflation lumen, a distal end, a proximal end, a proximal shaft section that is substantially rigid and a distal shaft section that is more flexible than the proximal shaft section. Also, the catheter shaft is dark in color. An inflatable balloon is disposed on the distal shaft section. The inflatable balloon is connected to the inflation lumen and the inflatable balloon has a non-circular cross-sectional shape when partially inflated. In this embodiment, the balloon dilation catheter includes a first proximal shaft marker disposed on the proximal shaft section, and the first shaft marker having a significantly lighter color than the catheter shaft. The first proximal shaft marker allows a user to approximate, using direct visualization of the first proximal shaft marker, a position of the balloon relative to a guide catheter through which the balloon catheter is advanced. There is also a first distal shaft marker disposed on the distal shaft section proximal to a proximal end of the balloon and the first distal shaft marker has a significantly lighter color than the catheter shaft. The first distal shaft marker enables a user to approximate, using endoscopic visualization of the first distal shaft marker, a position of the balloon relative to an opening of a paranasal sinus.

In one embodiment, a second proximal shaft marker is disposed on the proximal shaft section distally from the first proximal shaft marker and having a significantly lighter color than the catheter shaft. The first proximal shaft marker has a greater length than the second proximal shaft marker. Further, the length of the first proximal shaft marker is equal to the length from a proximal end of the inflatable balloon to the distal end of the catheter shaft. The first proximal shaft marker is spaced from the distal end of the catheter shaft such that it allows the user to approximate when the distal end of the catheter shaft is located at a distal end of the guide catheter and when the proximal end of the balloon exits a guide catheter, and wherein the second proximal shaft marker allows the user to approximate when the distal end of the catheter shaft is located just proximal to a curve in the guide catheter

The balloon dilation catheter device may also include a second distal shaft marker disposed on the distal shaft section proximal to the first distal shaft marker and having a significantly lighter color than the catheter shaft. The first distal shaft marker is disposed at a known distance proximally from the proximal end of the balloon, and the second distal shaft marker is disposed at a known distance proximally from the first distal shaft marker. Also, the first and second distal shaft markers have different appearances. In one embodiment, the first distal shaft marker is disposed approximately one centimeter from the proximal end of the balloon and the second distal shaft marker is disposed approximately two centimeters from the proximal end of the balloon. A third distal shaft marker also may be disposed on the distal shaft section at the proximal end of the balloon.

The balloon dilation catheter device may also include a first radiopaque marker disposed on the distal shaft section and within the inflatable balloon. There may be a second radiopaque marker disposed on the distal shaft section distally from the first radiopaque marker within the balloon. The first and second radiopaque markers are disposed a distance apart from one another to indicate the effective length of the inflatable dilator.

Also, in one embodiment, the inflatable balloon of the balloon dilator catheter device has an approximately triangular cross-section in a partially inflated state. The balloon may also have a balloon neck extending from the balloon proximally along the catheter shaft. The balloon neck allows an endoscopic marker to be disposed on the distal shaft section and underneath the balloon neck.

In an embodiment of a system for treating a disease or disorder of the ear, nose or throat of a human or animal subject, the system includes a guide catheter that is insertable into a head of the subject and has a substantially rigid shaft, a proximal opening, a distal opening and a lumen extending between the proximal opening and the distal opening. The system also includes a balloon catheter device as described above that is advanceable out of the distal opening of the guide catheter. The balloon catheter device also includes a guidewire lumen and the system includes a guidewire that is advanceable through the guidewire lumen. Also, the inflation lumen of the catheter shaft is sized so that, after the inflatable balloon has been inflated to a working diameter, the inflatable balloon will deflate in less than 5 seconds with application of negative pressure to the inflation lumen by a conventional balloon catheter inflation and deflation device.

The system may also include an irrigation catheter sized for advancement through the guide catheter into a paranasal sinus.

Furthermore, in an embodiment of a method for dilating a paranasal sinus ostium of a paranasal sinus of a patient, the method includes advancing a guide catheter into a head of a patient such that a distal end of the guide catheter is positioned within or near a paranasal sinus ostium of a paranasal sinus. By “paranasal sinus ostium,” it is meant the anatomical, non-manmade opening into the sinus ostium. A paranasal sinus ostium is formed by mucosal tissue overlying bone. For the purposes of this application the bone forming the paranasal sinus ostium is unfractured when initially addressed using the devices and methods described herein, although the described devices and methods may in some embodiments be used to fracture said bone. Also, the method includes inserting an endoscope into the patient's head and advancing a balloon catheter through a lumen of the guide catheter such that a balloon of the catheter passes out of the distal end of the guide catheter. With the endoscope, a first distal shaft marker disposed on a shaft of the balloon catheter a first known distance from the balloon may be viewed, and also, a second distal shaft marker disposed on the shaft a second known distance from the balloon may be viewed. The method may include approximating a location of the balloon relative to the paranasal sinus ostium, using the first and second distal shaft marker and their known distances from the balloon. The balloon of the balloon catheter may be expanded to remodel or break bone underlying mucosa of the paranasal sinus ostium and dilate the ostium.

The method may also include viewing a first proximal shaft marker during the step of advancing the balloon catheter. When a distal end of the first proximal shaft marker enters a proximal end of the guide catheter a distal end of the balloon catheter shaft is located approximately at the distal end of the guide catheter. Also, when a proximal end of the distal shaft marker enters the proximal end of the guide catheter a proximal end of the balloon of the catheter is located approximately at the distal end of the guide catheter.

Further, the method includes viewing a second proximal shaft marker during the step of advancing the balloon catheter. The second proximal shaft marker is disposed distal to the first proximal shaft marker, and when the second proximal shaft marker is located approximately at the proximal end of the guide catheter, the distal end of the balloon catheter is located immediately proximal to a curve in the distal end of the guide catheter. The first distal shaft marker is located proximal to the balloon and the second distal shaft marker is located distal to the balloon.

In another embodiment, the first and second distal shaft markers are located proximal to the balloon. The first distal shaft marker, may be located approximately one centimeter proximal to a proximal end of the balloon and the second distal shaft marker may be located approximately two centimeters proximal to the proximal end of the balloon. The method may also include viewing a third distal shaft marker located at the proximal end of the balloon.

The method may further include advancing a guidewire through the guide and through the ostium before advancing the balloon catheter. After the guidewire is in place, the balloon catheter is advanced over the guidewire and through the guide.

Also, the method may include removing the balloon catheter through the guide catheter and advancing an irrigation catheter through the guide catheter into the paranasal sinus. Once the irrigation catheter is in position, the sinus may be irrigated using the irrigation catheter.

Still further embodiments, aspects, features and details of the present invention will be understood upon reading of the detailed description and examples set forth herebelow.

### BRIEF DESCRIPTION OF THE DRAWINGS

FIG. 1 is a side view of one embodiment of a dilation catheter with its dilator in an expanded configuration.

FIG. 1A is a cross sectional view through line 1A-1A of FIG. 1 with an enlarged break-out view of a portion thereof.

FIG. 1B is an enlarged side view of the dilator and distal end of the dilation catheter of FIG. 1.

FIG. 2 shows a collection of transnasal guide catheters useable as components of a dilation catheter system.

FIG. 3 is a side view of one embodiment of a handle apparatus.

FIG. 3A is a side view of another embodiment of a handle apparatus.

FIG. 3B is a side view of yet another embodiment of a handle apparatus.

FIG. 3C is a side view of yet another embodiment of a handle apparatus.

FIG. 4 is an exploded, partial view of one embodiment of a dilation catheter system, including an optional handle apparatus.

FIG. 5 is a schematic diagram of one embodiment of a dilation catheter system (without the optional handle apparatus) being used to dilate the ostium of a paranasal sinus.

FIG. 6 is a schematic diagram of one embodiment of a dilation catheter system (with the optional handle apparatus) being used to dilate the ostium of a paranasal sinus.

FIG. 7A shows a partial view of the system of FIG. 5, including a guidewire stop/connector apparatus mounted on the guidewire prior to advancement of the guidewire.

FIG. 7A shows a partial view of the system of FIG. 5, including a guidewire stop/connector apparatus mounted on the guidewire and engaged with the hub of the dilation catheter following advancement of the guidewire.

FIG. 8A shows the dilation catheter system of FIG. 5 and an endoscope being held by one hand of the operator while the operator's other hand is being used to advance the guidewire of the system into a paranasal sinus.

FIG. 8B shows the dilation catheter system of FIG. 6 and an endoscope being held by one hand of the operator while the operator's other hand is being used to advance the dilation catheter so that its dilator becomes positioned within the ostium of the paranasal sinus.

FIG. 9 is a flow diagram showing steps in one method for using a dilation catheter system.

FIG. 10 is a flow diagram showing steps in another method for using a dilation catheter system.

FIG. 11 is a flow diagram showing steps in yet another method for using a dilation catheter system.

FIG. 12 is a side view of another embodiment of a dilation catheter with its dilator in an expanded configuration.

FIG. 12A is a cross sectional view through line 12A-12A of FIG. 12.

FIG. 12B is an enlarged side view of the dilator and distal end of the dilation catheter of FIG. 12.

### DETAILED DESCRIPTION

The following detailed description and the accompanying drawings are provided for the purpose of describing some, but not necessarily all, examples or embodiments of the invention. The contents of this detailed description and the accompanying drawings are exemplary in nature and do not limit the scope of the invention in any way.

### A First Embodiment of a Dilation Catheter

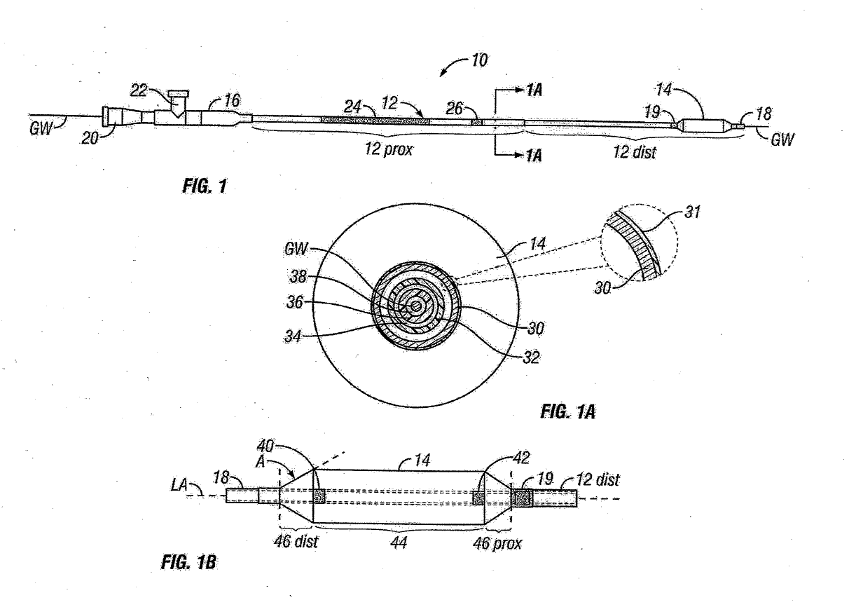

FIGS. 1-1B show one example of a dilation catheter device 10 of the present invention with a guidewire GW operatively inserted therethrough. In this example, the dilation catheter device 10 comprises an elongate catheter shaft 12 having a proximal shaft section 12prox that is substantially rigid and a distal shaft section 12dist that is more flexible than the proximal shaft section 12prox. An expandable dilator, such as a balloon 14 or other suitable mechanical or non-inflational dilator, is mounted on the distal shaft section 12dist and a distal tip member 18 protrudes beyond the distal end of the balloon 14, as shown. Also, a proximal T hub 16 is attached to the proximal end of the proximal shaft section 12prox. This proximal T hub 16 has a proximal Luer connector 20 and a side arm 22 having a female Luer connector that extends substantially perpendicular to the longitudinal axis of the hub 16, as shown. When compared to a typical Y hub, the side arm 22 of this T hub is further away from the proximal Luer connector 20 and is oriented at a right angle to the proximal Luer connector 20. Thus, tubing connected to this perpendicular side arm 22 is less likely to obscure or block the proximal Luer connector 20 than in a typical Y hub and the operator is less likely to confuse the proximal Luer connector 20 with the Luer connector on the side arm 22.

Although, in the particular example shown in the drawings, the expandable dilator comprises a balloon 14, it is to be appreciated that various other types of expandable dilators such as expandable cages, struts and other expandable mechanical assemblies may be used as an alternative to a balloon 14. Some non-limiting examples of expandable dilators other than balloons have previously been described in parent U.S. patent application Ser. No. 11/355,512, 11/150,874, 10/944,270 and 10/829,917, which are expressly incorporated herein by reference.

For use in teenage or adult humans, the overall length of the catheter shaft 12 may be in the range of about 15 cm to about 25 cm, the proximal shaft section 12prox may have a length in the range of about 10 cm to about 15 cm and the distal shaft section 12dist may have a length in the range of about 5 cm to about 10 cm. In the particular example shown in the drawings and described herein, the catheter shaft 12 has an overall length of 21.2 cm, the proximal shaft section 12prox being 12.5 cm in length and the distal shaft section 12dist being 8.7 cm in length. These optimal lengths of the proximal shaft section 12prox and distal shaft section 12dist have been arrived at based on a number of considerations, which will be discussed more fully herebelow in relation to the concurrent use of this dilation catheter 10 with a trans-nasal guide catheter.

As may be appreciated from the cross sectional view of FIG. 1A, the proximal shaft section 12prox comprises a rigid outer tube 30, a flexible middle tube 32 disposed substantially coaxially within the lumen of the rigid outer tube 30, and an inner tube 36 disposed substantially coaxially within the lumen of the middle tube 32. In this particular example, the outer tube 30 is formed of stainless steel hypotube having an outer diameter of 0.076 inches and an inner diameter of 0.068 inches. As an alternative to stainless steel hypotube, this outer tube 30 may be formed of rigid non-metallic material such as polyetheretherketone (PEEK) or other rigid plastics suitable for such application. Alternatively, other rigid reinforcing members may be used in, or in lieu of, the outer tube, such as wires (round, flat, square or of other cross section), partial tubes (e.g., arcs), etc. Also, in this particular example, the middle tube 32 is formed of Pebax having an inner diameter of 0.055 inches, an outer diameter of 0.065+/−0.003 inches. The inner tube 36 is formed of polyether block copolymer tubing (e.g., Pebax® Resin, Arkema, Inc., Philadelphia, Pa.) having an inner diameter of 0.038 inches, an outer diameter of 0.048 inches.

The outer tube 30 terminates at the end of the proximal shaft section 12prox. The middle tube 32 and inner tube 36 extend beyond the distal end of the outer tube 30, forming the distal shaft section 12dist.

As seen in the enlarged break-out segment of FIG. 1A, a polyether block copolymer film laminate 31 (e.g., Pebax® Resin, Arkema, Inc., Philadelphia, Pa.) is heat shrunk onto the outer surface of the catheter shaft 12 from the proximal hub 16 to the balloon 14. This laminate 31 provides a smooth outer surface and smoothes the step-down in diameter from the distal end of the proximal shaft section 12prox to the proximal end of the distal shaft section 12dist (i.e., it provides a smooth surface over the distal end of the outer tube 30 and the adjacent outer surface of the middle tube 32). The smooth step down may also be formed by an adhesive fillet. In other embodiments, the smooth step down may be formed by tapering or chamfering the structure of the distal end of the proximal shaft, eliminating the need for a laminate or adhesive.

The proximal end of the middle tube 32 extends into and is secured to the hub 16, distal to side arm Luer connector 22. The proximal end of the inner tube 36 extends into and is secured within hub 16, proximal to the side arm Luer connector 22 and in direct alignment and fluid communication with proximal Luer connector 20. The distal end of the middle tube 32 terminates within the balloon 14 and the proximal end of the dilator is secured to the outer surface of the middle tube. The distal end of the inner tube 36 also extends through the balloon 14 and protrudes distally beyond the balloon 14, forming the relatively flexible distal tip member 18 as shown in FIG. 1. The distal end of the balloon 14 is secured to the outer surface of the inner tube 36. In this manner, the inner tube lumen 38 extends through the entire catheter shaft 12 from the proximal Luer connector 20 through the distal tip 18 and may be used a guidewire lumen or as a working lumen for infusion of irrigation solution, medicaments, contrast media or other substances and/or for aspiration of blood, fluids or debris. Guidewires that may be advantageously used in conjunction with this dilation catheter 10 may have a length of 60 cm to 80 cm and may be either 0.014 inch or 0.035 inch, such as those commercially available as the Relieva® Sinus Guidewires (Acclarent, Inc., Menlo Park, Calif.) or sizes in between such as 0.018 inch, 0.020 inch, or 0.033 inch. Although the drawings show an over-the-wire catheter having a guidewire lumen that extends through the entire length of the catheter, it is to be appreciated that guidewire lumens extending less than the entire length of the catheter (e.g., rapid exchange guidewire lumens) may be used as an alternative to the over-the-wire lumen shown. Additionally, in some embodiments, rather than advancing the catheter over a guidewire, the catheter may be equipped with a fixed guidewire tip such as any of those described in U.S. patent application Ser. No. 11/438,090 entitled Catheters with Non-Removable Guide Members Useable for Treatment of Sinusitis, the entire disclosure of which is expressly incorporated herein by reference.

The inner tube lumen 38 may be lined or coated with a lubricious material to facilitate passages of the guidewire GW through that lumen 38. The diameter of the inner tube 36 may be changed to accommodate guidewires of different diameter. In the particular embodiment described, the inner tube lumen 38 is sized to receive a 0.035 inch diameter guidewire GW. The inner tube lumen 38 may be internally lined or coated with a 2% solution of linear polydimethylsiloxane (PDMS) (e.g., Dow Corning® 360 Medical Fluid, Dow Corning Corporation, Midland, Mich.) diluted in isopropyl alcohol or another silicone material (such as a 2% solution of Dow-Corning MDX4-4159 in isopropyl alcohol). The coating is cured at room temperature.

The luminal space 34 between the outer surface of the inner tube 36 and the inner surface of the middle tube 32 is in fluidic communication with the side arm Luer connector 22 and extends to the interior of the balloon 14. Thus, this luminal space 34 serves as the passageway through which inflation fluid passes into and out of the balloon 14. The size of this luminal space 34 and the relatively short length of the catheter shaft 12 are optimized to minimize drag on inflation fluid passing through this luminal space 34 and allow for rapid deflation of the balloon 14. The clearance of 0.006 to 0.007 inches between the inner and outer members is desired for catheter length of 20-35 cm. The desired deflation time is 5-10 seconds, and the deflation time is measured with application of negative pressure on the inflation/deflation lumen using a 20 cc inflation device that is filled with 10 cc contrast/saline mixture.

Balloon Construction and Coating

FIG. 1B shows details of the balloon 14. In this example, the balloon 14 is a non-compliant balloon formed of polyethylene terephthalate (PET) film having a thickness of 0.8 mm. The balloon 14 has a cylindrical mid-region 44 and tapered proximal and distal end regions 46prox and 46dist. The balloon 14 has an overall length of 2.6 cm. The cylindrical midregion 44 of the balloon 14 has a length of 16 mm (i.e., the “working length”) and each tapered end region 46prox, 46dist has a length of 5 mm. The balloon 44 has a burst pressure of at least 14 to 16 atmospheres. The outer diameter of the balloon 14, when inflated to a pressure of 14 atmospheres, may be in the range of 5.0 mm to 5.5 mm. In this particular example, the balloon 14 is sized for dilation of the ostia of paranasal sinuses, and such balloon 14 is offered in sizes having outer diameters of 5 mm or 7 mm when inflated to a pressure of 14 atmospheres. Dilation catheters 10 having the 5 mm diameter balloon 14 may be more suitable for use in subjects of small body size while dilation catheters 10 having the 7 mm diameter balloon 14 may be more suitable for use in subjects having a large body size. Smaller or larger balloons may be used for dilating structures other than the ostia of paranasal sinuses (e.g., Eustachian tube or naso-lacrimal duct dilations). Larger balloons and higher pressures may be used for dilating revision patients (i.e., patients who have had prior ostial dilations or whose ostia have been previously modified by surgery).

The tapered end regions 46prox, 46dist are tapered at angle A relative to the longitudinal axis LA of the catheter shaft 12 on which the balloon 14 is mounted. This angle of taper A may be in the range of about 10 degrees to about 30 degrees. In the particular example shown in the drawings, such angle of taper A is 20 degrees. This 20 degree angle of taper provides an improved transition from the balloon working length to the balloon necks, lower profile, improved crossing, improved tracking, and easier withdrawal of the balloon into the sinus guide catheter after balloon inflation and deflation. It also provides optimal performance with minimum increase of overall balloon length.

In some embodiments, it may be desirable for the relatively stiff proximal shaft portion 12prox to extend all the way to or near the proximal end of the balloon 14 or other dilator. Such catheter having a rigid shaft from its proximal end to or near the dilator may be advanced directly into the sphenoid sinus ostium with or without the use of a guide catheter. In some embodiments, the proximal end of the balloon 14 could be bonded to the relatively rigid proximal shaft portion 12prox. Such a construction would allow the flexible distal tip 18 to track turns in the anatomy and may be useable to dilate certain passageways (e.g., the sphenoid sinus ostium) without disrupting the normal anatomy. Additionally, embodiments with relatively short distal shaft sections (e.g., 1-2 cm beyond the distal end of the rigid proximal shaft portion) are particularly suitable for dilating the ostia of frontal sinuses. Also, in some embodiments, the proximal shaft section 12prox may be malleable so that it may be shaped (e.g., bent or formed to a desired curve or multi-curve shape) to facilitate access to any desired passageways or locations.

Endoscopically Visible Markers and Anti-Glare Coatings

In some embodiments, a visible distal shaft marker 19 may optionally be placed adjacent the proximal end of the balloon 14 and/or on the distal shaft portion 12dist, such as at the location where the proximal end of the balloon 14 is bonded to the distal shaft portion 12dist. Additionally or alternatively, one or more proximal shaft markers 24, 26 may also be placed along the proximal shaft portion 12prox. Generally, the distal shaft marker 19 is positioned to be viewed using an endoscope while the distal shaft portion 12dist resides within a patient, and thus the marker 19 may be referred to as an “endoscopic marker.” The other visible markers 24, 26, formed on the proximal shaft portion 12prox, are specifically designed for use in conjunction with a guide catheter, as will be discussed in detail below.

In one embodiment, these visible markers 19, 24, 26 are preferably of a color (e.g., black or blue) that contrasts with the pink color of the nasal mucosa so as to be easily visible within the nose. In an alternative embodiment, the catheter shaft 12 may be of a dark color, and the markers 19, 24, 26 may be of a light color, so that their contrasting colors facilitate visualization of the markers 19, 24, 26. The optional marker 19 on the proximal end of the balloon 14 allows the operator to endoscopically view the proximal end of the balloon even when the remainder of the balloon is within the ostium of a paranasal sinus.

In some cases, endoscopic images obtained of the marker 19, other portions of the dilation catheter 10, a guidewire GW and/or a guide catheter 70a-70f used in a dilation procedure may have areas of glare, which can obscure visualization of certain portions of the endoscopic marker 19 or devices during performance of the procedure. To minimize such glare, an anti-glare (e.g., anti-reflective) treatment or coating may be applied to all or part of the sinus guide catheter 70a-70f, sinus guidewire GW and/or dilation catheter 10. Such anti-glare treatment could be applied by etching or sand-blasting and therefore does not add profile to the device. Such anti-glare coating could be applied by dip or spray coating and is very thin. The treatment or coating does not change the mechanical or functional properties of these devices. It may be selectively applied. For example, a black polytetrafluoroethylene (PTFE) coating on the sinus guidewire GW may provide good anti-reflective characteristics. Some of the commercially available anti-glare or anti-reflective coating can be applied. In some embodiments, an anti-glare surface treatment (e.g., roughening, etching, etc.) may be used or an anti-glare component such as a sheath, ring, paint, etc. may be used.

The advantages and benefits of including visible markers and/or the anti-glare coating include, improved endoscopic visualization, safer and easier performance of the procedure, reduced balloon burst or damage to critical structures, accuracy of placement of devices and reduced fluoroscopy time or elimination of fluoroscopy.

Dilation Catheter/Guide Catheter System

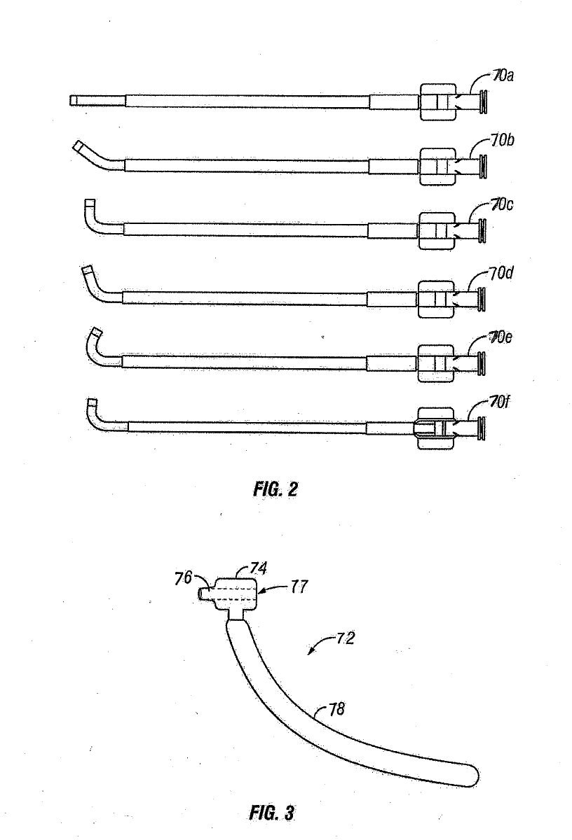

FIG. 2 shows a series of sinus guide catheters 70a-70f that may be used in conjunction with the dilation catheter 10. These guide catheters 70a-70f are substantially rigid and each has a preset distal curve of 0 degrees (70a), 30 degrees (70b), 90 degrees (70d), 70 degrees (70c) or 110 degrees (70e and 70f). Different curvatures are useable to access the ostia of different sinuses. For example, a 70 degree guide is typically used to access the ostium of a frontal sinus, a 90 or 110 degree guide is typically used to access the ostium of a maxillary sinus, etc. Each of these guide catheters 70a-70f has a length of 12.7 cm. These sinus guide catheters are described in parent U.S. patent application Ser. Nos. 11/355,512, 11/150,874, 10/944,270 and 10/829,917, which were previously incorporated by reference, and are now commercially available as Relieva® sinus guide catheters from Acclarent, Inc., Menlo Park, Calif.

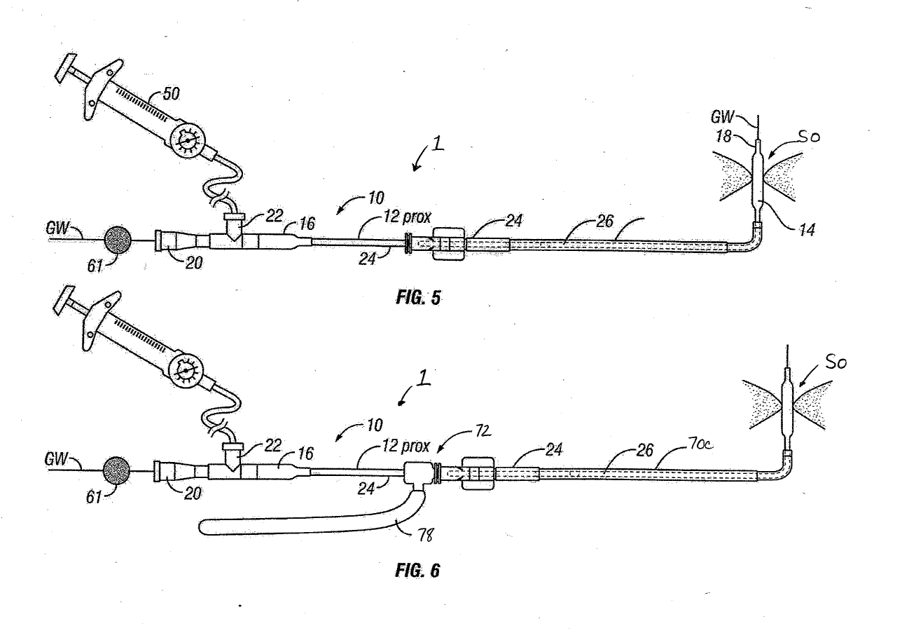

FIG. 5 shows a system 1 comprising a guide catheter 70c having a 90 degree curve formed therein in combination with a dilation catheter 10 shown in FIG. 1. In optimizing the relative lengths of the proximal shaft section 12prox and distal shaft section 12dist, applicants have determined that the maximum distance that the distal end of the dilation catheter 10 is required to travel beyond the distal end of the guide catheter 70c is approximately 2.5 cm. However, it will be appreciated that this is just one example. For other applications, travel beyond 2.5 cm may be desirable or necessary. Also, it is desirable for the more flexible distal shaft section 12dist to have a length such that it may reside within the guide catheter 70a-70f proximal to any curve formed in the guide catheter. With these objectives in mind, the example of the dilation catheter 10 shown in the drawings has a shaft that is about 20 cm in length, with the proximal shaft section 12prox being 11.3 cm in length and the distal shaft section 12dist being 8.7 cm in length. Thus, prior to or during the procedure, the entire distal shaft section 12dist of the dilation catheter 10 may be initially advanced into the rigid guide catheter 70c without the distal portion of the dilation catheter 10 passing through the curve of the guide catheter 70c and with only a portion of the rigid proximal shaft section 12prox of the dilation catheter 10 protruding out of the proximal end of the guide catheter 70c. To facilitate such positioning of the dilation catheter 10 within the guide catheter 70c, and with reference again to FIG. 1, a first shaft marker 26 is provided on the proximal shaft section 12prox of the dilation catheter shaft 12. The distal edge of this first shaft marker 26 is 2.7 cm proximal to the distal end of the proximal shaft section 12prox and 11.4 cm from the distal end of the distal tip member 18. If the operator advances the dilation catheter 10 into the guide catheter 70c until the distal edge of the first shaft marker 26 is flush with the proximal end of the guide catheter 70c, the entire distal shaft portion 12dist as well as the distal-most 3 cm of the proximal shaft portion 12prox will be housed within the guide catheter 70c such that the distal end of the dilation catheter 10 is located proximal to the curve formed near the distal end of the guide catheter 70c. Such positioning of the dilation catheter 10 within the guide catheter 70c provides a guide catheter/dilation catheter assembly that is substantially rigid from the proximal hub 16 of the dilation catheter 10 to the distal end of the guide catheter 70c. As a result, the operator may hold or support the entire assembly by grasping or supporting just one location on either the dilation catheter 10 or guide catheter 70c. For example, the user may hold or support the entire assembly by using his fingers to grasp or support either the proximal hub of the guide catheter 70c, the proximal hub 16 of the dilation catheter 10, somewhere on the proximal shaft section 12prox of the dilation catheter, or on the shaft of the guide catheter 70c. Such rigidity also substantially eliminates the potential for the exposed portion of the dilation catheter 10 to droop down onto the subject's chest or onto the adjacent operating table.

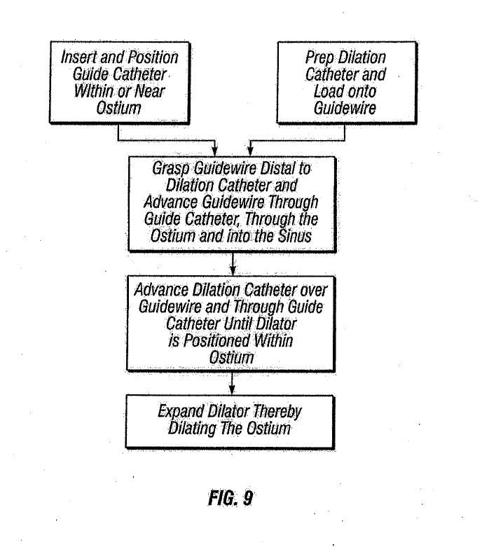

As explained above, in this example, the rigid proximal shaft segment 12prox of the dilation catheter 10 is 11.3 cm in length and the guide catheter 70c is 12.7 cm in length. Thus, when inserted into the subject's body, the overall length of the portion of the system that remains exposed (e.g., the proximal part of the guide catheter 10 extending out of the subject's nose and the proximal part of the dilation catheter 10 extending out of the proximal end of the guide catheter 70c) is not only rigid, but sufficiently short (e.g., typically less than 9 cm) to be easily manageable and capable of being held or supported by a single hand of the operator, thereby allowing the operator's other hand to be used for other purposes, such as for advancing/retracting the guidewire GW or advancing/retracting the dilation catheter 10 in the manner described below in connection with FIGS. 9-11.

The second shaft marker 24 correlates to the position of the balloon. If the dilation catheter 10 is advanced to a position where the distal edge of the second shaft marker 24 is flush with the proximal end of the guide catheter 10, the distal tip of the balloon catheter will be flush with the distal tip of the guide catheter 70c. When the proximal edge of the second shaft marker 24 is flush with the proximal end of the guide catheter 10, the entire balloon 14 will have advanced out of the distal end of the guide catheter 70c, and the operator will know that it is safe to inflate the balloon. Typically, as seen in FIG. 5, the balloon 14 is advanced some distance out of the distal end of the guide catheter 70c until the balloon 14 is positioned within the sinus ostium SO or other passageway to be dilated. As seen in the enlarged view of the balloon 14 shown in FIG. 1B, proximal and distal radiographic markers 40, 42 are provided on the catheter at either end of the cylindrical segment 44 of the balloon. A C arm fluoroscope may be positioned and used to image those proximal and distal markers 40, 42 as well as the sinus ostium SO, and the position of the dilation catheter 10 may be adjusted as needed until the sinus ostium SO is midway between the proximal and distal radiographic markers 40, 42. Thereafter, an inflator 50 attached to the side arm Luer connector 22 may be used to inflate the balloon 14, thereby dilating the sinus ostium SO as shown in FIG. 5. In keeping with the operator's ability to use a single hand to hold or support the exteriorized portion of the system, the inflator 50 may be attached to the side arm Luer connector 22 in advance and may be controlled by a foot pedal which is actuated by the operator's foot.

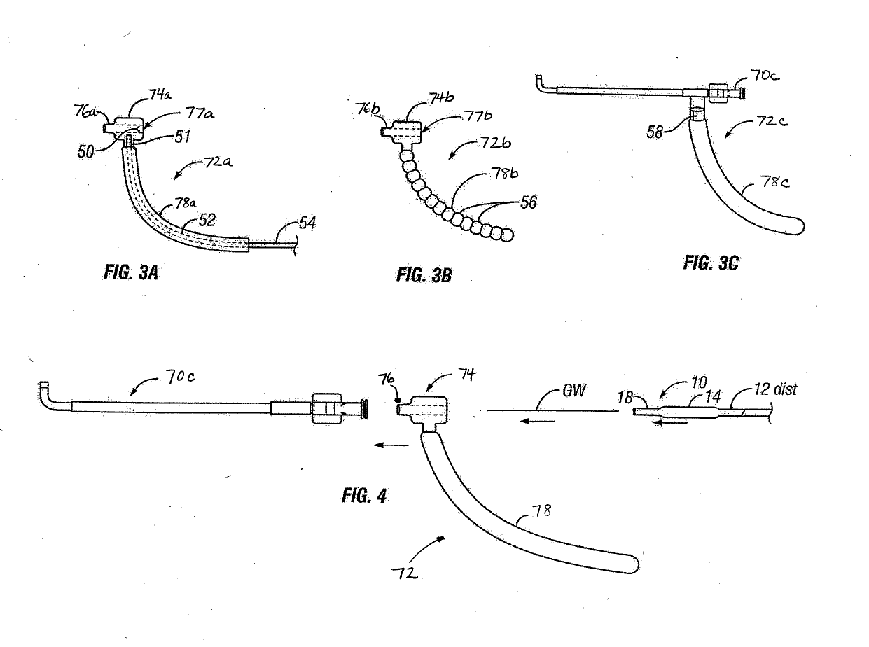

In some applications of the system 1 shown in FIG. 5, an endoscope may be placed in the nose and used to view all or part of the procedure. Because the exposed portion of the system 1 is substantially rigid and is typically less than 15 cm in length, the operator may use a single hand to hold the endoscope as well as the dilation catheter/guide catheter system 1. Alternatively, a scope holder may be used to hold the endoscope in a fixed position while the operator positions and uses the system 1. Alternatively, an optional handle may be used as shown in FIGS. 3-4, 6 and 8A-8B and described below.

Optionally, a member 61 may be attached to the guidewire. Such member may serve to prevent the dilation catheter 10 and/or guide catheter 70c from inadvertently sliding off of the proximal end of the guidewire. Also, such member 61 may limit the length of guidewire GW that may be advanced through the dilation catheter 10. This will prevent the operator from advancing too much of the guidewire GW into the subject's sinus, which may injure or damage the mucosa lining the sinus cavity. In some embodiments, this member 61 may be a standard guidewire torquer of the type commercially available and well known in the fields of interventional cardiology and/or radiology. One example of a commercially available guidewire torquer that is useable with the catheter 10 in this application is a two part torquer available as Part No. 97333 from Qosina, Corp., Edgewood, N.Y.

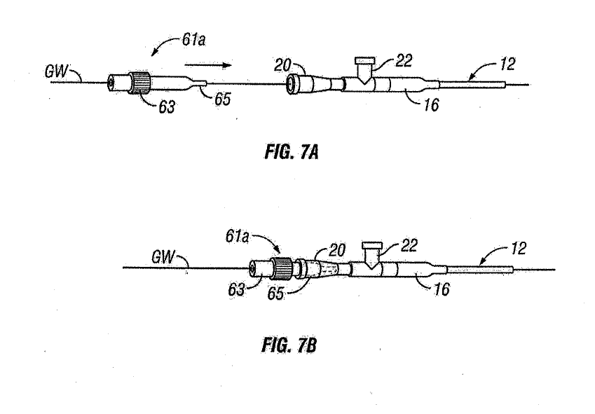

Alternatively, the member 61 may comprise a guidewire stop/connector apparatus 61a as shown in FIGS. 7A-7B. This stop/connector apparatus 61a comprises a rigid plastic body 63 having a lumen extending therethrough and a tapered elastomeric tube member 65 on its distal end. The stop/connector apparatus 61a is advanced over the guidewire GW to the desired location. The inner diameter of the tapered elastomeric tube member 65 fits snugly on the guidewire, thereby holding the stop/connector apparatus 61a as seen in FIG. 7A. The guidewire GW is subsequently advanced through the dilation catheter 10 until the tapered elastomeric tube member 65 is received within and frictionally engages the proximal female Luer connector 20 on the hub of the dilation catheter, as shown in FIG. 7B. This limits advancement of the guidewire GW and also frictionally locks the guidewire GW to the dilation catheter 10 so that the operator may move both the guidewire GW and the dilation catheter 10 as a unit. If the operator decides to advance more of the guidewire into the sinus, the operator may grasp and move the stop/connector apparatus 61a by applying sufficient force to overcome the frictional engagement between the stop/connector apparatus 61a and the guidewire GW and/or between the stop/connector apparatus 61a and the guide catheter hub. The force required to overcome such frictional engagements will preferably be greater than the forces that would normally result form routine movement and use of the system, thereby allowing the stop/connector apparatus 61a to perform its locking function while still allowing the location of the stop/connector apparatus 61a to be volitionally adjusted by the operator when necessary.

Alternatively or additionally, if desired, another stop/connector apparatus 61a of larger size (or another suitable locking apparatus such as a Touhy-Borst valve) may be mounted on the rigid proximal shaft section 21prox of the dilation catheter 10 and received within the proximal end of the guide catheter 70a-70f to limit the advancement of the dilation catheter 10 through the guide catheter 70a-f and to frictionally lock the dilation catheter 10 to the guide catheter 70a-f in the same manner.

Dilation Catheter/Guide Catheter System with Optional Handle

FIG. 3 shows an optional handle 72 that may be attached to the guide catheter 70a-70f to facilitate single-handed holding of the guide catheter/dilation catheter system as well as an endoscope (or other device). The handle 72 shown in FIG. 3 comprises a rigid head 74 having a male Luer fitting on one end, a lumen 77 extending therethrough and a handle member 78 extending therefrom. As seen in the exploded view of FIG. 4, the male Luer fitting 76 may be inserted into the proximal end of the guide catheter 70c, and the guidewire GW and guide catheter 10 may then be inserted through the lumen 77 of the handle head 44 and through the guide catheter. The handle head 74 may be clear or transparent, so that the operator may view the shaft markers 24, 26 on the dilation catheter shaft 12 as the dilation catheter 10 is advanced through the handle head 74. Alternatively, the locations of the shaft markers 24, 26 may be adjusted on the catheter shaft 12 to take into account the additional guide length added by the handle head 74. The handle member 78 is preferably about the size of a standard ink pen and may be conveniently grasped by a human hand. The handle member 78 may have a roughened or elastomeric surface to facilitate gripping by a gloved hand and to deter slippage of the handle from the operator's grip. The handle member 78 may be shapeable (e.g., malleable or bendable) to allow the operator to adjust the shape and/or angle of the handle relative to the shaft of the guide catheter 70c. In some embodiments, the handle member 48 may be pre-shaped to accommodate a typical user and allow fine tuning by an individual user. Also, in some embodiments, the handle member 78 may have foam or other material on its surface to facilitate grip. The handle member 78 may have various different cross sectional profiles (e.g., round, oval, 3-sided, 4-sided, 5-sided, 6-sided, etc.). The handle member 78 serves to facilitate grip and control to manipulate the dilation catheter along with a separate device (e.g., an endoscope or other tool) without having to use a second hand. In this manner, the user may adjust rotation of a guide catheter while observing via an endoscope (all with one hand) and use other hand to advance and place the guidewire or other device. Also, in some embodiments, the handle member 78 may include finger loop(s) for easier and/or more secure handling. Also, in some embodiments, a pinch valve or hole can be strategically placed in the handle member 78 to actuate/allow control of suction or fluid delivery via the handle 72 (e.g., the user may pinch the handle with fingers to restrict flow through handle), or the handle 72 may have a suction hole where the user must cover the suction hole to actuate suction.

Alternative embodiments of the handle are shown in FIGS. 3A, 3B and 3C. FIG. 3A shows a handle 72a similar to that seen in FIG. 3, but wherein a fluid channel 52 extends from the lumen 77a downwardly through the head 74a and through the handle member 78a. A one way valve 50 is disposed within the lumen 77a, proximal to the location where the fluid channel 52 meets the lumen 77a. An irrigation and/or suction tube 54 may be attached to the handle member 48a to infuse fluid through or suction fluid and debris through the fluid channel 52. The one way valve will ensure that fluid infused or aspirated through the fluid channel 52 of the handle 72a will not escape out of the proximal opening of the lumen 77a. However, this one way valve 50 does allow the guidewire GW and dilation catheter 10 to be inserted through the lumen 77, when desired. The one way valve may provide the additional benefit of maintaining the position of the guidewire or dilatation catheter when it is inserted in the guide handle 72a. Other types of valves than a one-way valve may be used as an alternative (e.g., Touhy rotating type valve, slide to compress valve, etc.) Alternatively, some embodiments may simply include a valve and a thumb/finger hole to control the suction force as described above.

FIG. 3B shows another embodiment of an optional handle 72b comprising a clear or transparent rigid head 74b having a male Luer fitting 76b on one end and a lumen 77 extending therethrough. In this embodiment, the handle member 78b is formed of a series of pivotally interconnected units 56, which allows the handle member 78b to be conveniently formed into various shapes as desired by the operator.

FIG. 3C shows yet another handle 72c comprising a malleable or rigid handle member 78c that is substantially the same as that shown in FIG. 3, but wherein a clip 58 is provided at the top end of the handle member 78c to clip the handle member 78c onto the shaft of the guide catheter 70c rather than inserting into the proximal end of the guide catheter.

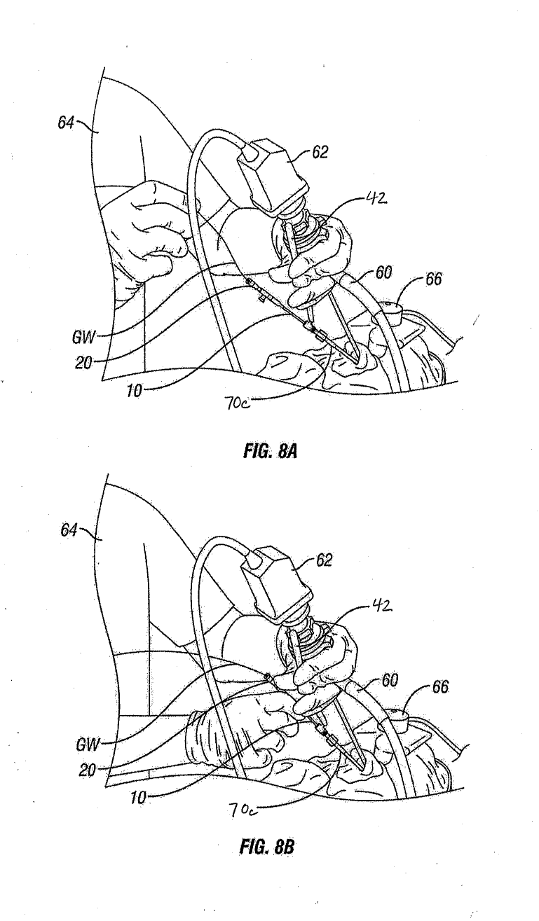

FIG. 6 shows the system of FIG. 5 with the inclusion of the optional handle 72 on the proximal end of the guide catheter 70c. FIGS. 8A and 8B show examples of how a handle 72 may be used to facilitate concurrent holding of an endoscope as well as the guide catheter (or guide catheter/dilation catheter assembly) by a single hand (i.e., the “scope hand”) of the operator. In one embodiment, the handle head 74 may initially be loosely inserted into the proximal hub of the guide catheter 70c. The camera 62 and light cable 66 are attached to the endoscope 60. While grasping the endoscope 60 in the manner shown in FIG. 8A, the operator may rotate the handle 72 relative to the guide catheter 70c to introduce the handle member 78 to the operator's scope hand. Alternatively, the handle member 78 could be grasped by the operator's scope hand along with the endoscope 60 upon initial introduction. When positioning of the endoscope 60 and guide catheter 70c have been achieved, the operator's other hand is used to push the male Luer fitting 76 of the handle 72 firmly into the female Luer fitting on the proximal end of the guide catheter 70c, thereby locking the handle 72 to the guide catheter 70c. Thereafter, the operator's other hand is used to manipulate the guidewire GW and dilation catheter 10. In this manner, the operator may maintain continuous endoscopic visualization via the endoscope 60 while using the guidewire GW and dilation catheter to dilate the ostium of a paranasal sinus or other passageway within the ear, nose or throat. As explained in more detail below, positioning of the guidewire GW and/or balloon 14 (or other dilator) may be confirmed using fluoroscopy, trans-illumination or other techniques in addition to visualization via the endoscope 60. The guide handle 72 may also be used to allow the operator to hold or support the guide catheter 70c (or the entire guide catheter/dilation catheter system) while keeping his hand spaced away from the guide catheter shaft to avoid radiation exposure to his hand during use of the fluoroscope.

In embodiments where the handle member 78 is shapeable (e.g., malleable or bendable), the shape of the handle member 78 may be modified one or more times prior to or during the procedure to facilitate comfortable grasping of the handle by the operator's scope hand and/or to adjust the position or angle of the endoscope relative to the guide catheter. In this regard, in FIG. 8A, the handle member 78 is bent to a shape that results in an angle between the shaft of the guide catheter 70c and the endoscope 60, and the operator's other hand is being used to advance the guidewire GW through the lumen of the dilation catheter 10. In FIG. 8A, the handle has been modified to a different shape that results in a lesser angle between the shaft of the guide catheter 70c and the endoscope 60, and the operator's other hand is being used to advance the dilation catheter 10 through the lumen of the guide catheter 70c.

The optional handle 72 may also be useful with other dilation catheters and other trans-nasal devices described in any or all of the parent applications of which this application is a continuation-in-part and/or those currently available commercially under the trademark Relieva from Acclarent, Inc., Menlo Park, Calif.

In some applications, the handle 72 may be designed to connect by way of a unique or proprietary connector to the guide catheter or other device. Alternatively, in some embodiments, the handle 72 may be pre-attached, integrally formed with or otherwise designed as a part or portion of the guide catheter or other device. In embodiments where the handle 72 is not detachable from the guide catheter or other device, it may nonetheless be rotatable and/or lockable in a desired position

Modes of Use of the System

FIGS. 9-11 are flow diagrams describing three modes of use by which the dilation catheter system described herein may be used to dilate the ostium of a paranasal sinus.

Mode 1—Inserting Guide Catheter, Guidewire and Dilation Catheter Separately

In the example of FIG. 9, the dilation catheter 10 is prepared for use separately from the guide catheter 70a-70f. The guide catheter 70a-70f is initially inserted (along with an endoscope 60) and is advanced to a position that is within or near the ostium to be dilated. An endoscope 60 is used to view the advancement and positioning of the guide catheter 70a-70f, and fluoroscopy may also be used to verify that the guide catheter is properly positioned near or within the ostium. Optionally, a handle 72 may be attached to the guide catheter 70a-70f as described above or the operator may simply grasp the guide catheter 70a-70f as well as the endoscope 60 with the scope hand, thus leaving the operator's other hand free to be used for subsequent handling and manipulation of the other devices used in this procedure. Alternatively, a scope holder or assistant may be used to hold the endoscope 60 in the desired position, thus freeing both of the operator's hands for handling and manipulation of the other devices.

After the guide catheter 70a-70f has been positioned, the operator will insert the distal end of the guidewire into the proximal end of the guide catheter 70a-70d and will advance the guidewire GW through the guide catheter 70a-70d such that a distal portion of the guidewire GW passes through the sinus ostium and becomes coiled within the sinus cavity. Fluoroscopy (or any other suitable technique) may be used to verify that the guidewire has become coiled within the intended sinus cavity.

Thereafter, the proximal end of the guidewire GW is inserted into the distal end of the dilation catheter 10, and the dilation catheter 10 (with its balloon 14 or other dilator in its non-expanded state) is advanced over the guidewire and through the guide catheter 70a-70d to a position where the dilator 14 is positioned within the sinus ostium. The endoscope 60 may be used to view the advancement and positioning of the dilation catheter 10. Although the distal portion of the balloon 14 or other dilator will be within the sinus and out of the field of view of the endoscope 60, the endoscope 60 may be used to view the proximal end of the balloon 14 or other dilator and/or the optional marker 19 (if present) on the proximal end of the balloon 14 or other dilator. Fluoroscopy may be used to image the radiographic markers 40, 42 and the ostium to confirm that the mid-region 44 of the balloon 14 (or the appropriate portion of any other type of dilator) is positioned within the ostium.

After the balloon 14 or other dilator has been positioned within the ostium, the balloon is inflated (or the other dilator is expanded) thereby dilating the ostium.

The balloon is then deflated (or the dilator is returned to its non-expanded state) and the successful dilation of the ostium may be confirmed visually using the endoscope 60 and/or radiographically using a fluoroscope.

Thereafter, the dilation catheter 10, guidewire GW and guide catheter 70a-70f are removed.

Mode 2—Preloading Dilation Catheter into Guide Catheter then Inserting Guidewire Separately

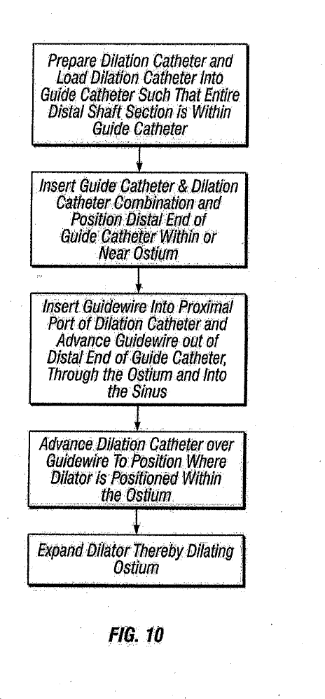

In the example of FIG. 10, the dilation catheter 10 is prepared for use and is pre-inserted into the guide catheter 70a-70f to a position where the first shaft marker 24 is flush with the proximal end of the guide catheter. When so positioned all of the flexible distal shaft portion 12dist and a bit of the rigid proximal shaft portion 12prox will be within the guide catheter 70a-70f.

Thereafter, the guide catheter 70a-70f in combination with the pre-inserted dilation catheter 10 is inserted transnasally (along with an endoscope 60) and is advanced to a position that is within or near the ostium to be dilated. The endoscope 60 is used to view the advancement and positioning of the guide catheter 70a-70f, and fluoroscopy may also be used to verify that the guide catheter is properly positioned near or within the ostium. Optionally, a handle 72 may be attached to the guide catheter 70a-70f, as described above, or the operator may simply grasp the guide catheter 70a-70f as well as the endoscope 60 with the scope hand, thus leaving the operator's other hand free to be used for subsequent handling and manipulation of the other devices used in this procedure. Alternatively, a scope holder or assistant may be used to hold the endoscope 60 in the desired position, thus freeing both of the operator's hands for handling and manipulation of the other devices.

After the guide catheter 70a-70f and pre-inserted dilation catheter 10 have been positioned, the operator will insert the distal end of the guidewire GW into the proximal Luer 20 of the dilation catheter 10 and will advance the guidewire GW through the dilation catheter 10, out of the distal end of the guide catheter 70a-70f and through the sinus ostium, causing a distal portion of the guidewire GW to become coiled within the sinus cavity. Fluoroscopy (or any other suitable technique) may be used to verify that the guidewire GW has become coiled within the intended sinus cavity.

Thereafter, the dilation catheter 10 (with its balloon 14 or other dilator still in its non-expanded state) is advanced over the guidewire GW to a position where the balloon 14 or other dilator is positioned within the sinus ostium. The endoscope 60 may be used to view the advancement and positioning of the dilation catheter 10. Although the distal portion of the balloon 14 or other dilator will be within the sinus and out of the field of view of the endoscope 60, the endoscope 60 may be used to view the proximal end of the balloon 14 or other dilator and/or the optional marker 19 (if present) adjacent the proximal end of the balloon 14 or other dilator. Fluoroscopy may be used to image the radiographic markers 40, 42 and the ostium to confirm that the midregion 44 of the balloon 14 (or the appropriate portion of any other type of dilator) is positioned within the ostium.

After the balloon 14 or other dilator has been positioned within the ostium, the balloon is inflated (or the other dilator is expanded) thereby dilating the ostium.

The balloon is then deflated (or the dilator is returned to its non-expanded state) and the successful dilation of the ostium may be confirmed visually using the endoscope 60 and/or radiographically using a fluoroscope.

Thereafter, the dilation catheter 10, guidewire GW and guide catheter 70a-70f are removed.

Mode 3—Preloading Guidewire and Dilation Catheter into Guide Catheter

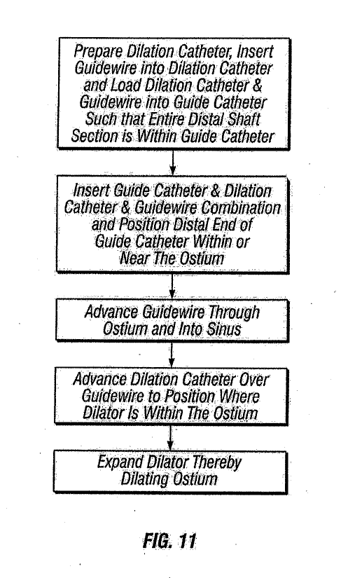

In the example of FIG. 11, the dilation catheter 10 is prepared for use and the distal end of the guidewire GW is pre-inserted into the proximal Luer 20 of the dilation catheter 10 and advanced to a position where the distal end of the guidewire GW is protruding just slightly out of the distal end of the dilation catheter 10. The dilation catheter 10, with the pre-inserted guidewire GW, is pre-inserted into the guide catheter 70a-70f and advanced to a position where the first shaft marker 24 is flush with the proximal end of the guide catheter 70a-70f. When so positioned, all of the flexible distal shaft portion 12dist and a bit of the rigid proximal shaft portion 12prox will be within the guide catheter 70a-70f.

Thereafter, the guide catheter 70a-70f with the dilation catheter 10 and guidewire pre-inserted therein is inserted through a nostril (along with an endoscope 60) and is advanced to a position that is within or near the ostium to be dilated. The endoscope 60 is used to view the advancement and positioning of the guide catheter 70a-70f, and fluoroscopy may also be used to verify that the guide catheter 70a-70f is properly positioned near or within the ostium. Optionally, a handle 42 may be attached to the guide catheter 70a-70f, as described above, or the operator may simply grasp the guide catheter 70a-70f as well as the endoscope 60 with the scope hand, thus leaving the operator's other hand free to be used for subsequent handling and manipulation of the other devices used in this procedure. Alternatively, a scope holder or assistant may be used to hold the endoscope 60 in the desired position, thus freeing both of the operator's hands for handling and manipulation of the other devices.

After the guide catheter 70a-70f and pre-inserted dilation catheter 10 and guidewire GW have been positioned, the operator will advance the guidewire GW out of the distal end of the guide catheter 70a-70f and through a sinus ostium, causing a distal portion of the guidewire GW to become coiled within the sinus cavity. Fluoroscopy (or any other suitable technique) may be used to verify that the guidewire GW has become coiled within the intended sinus cavity.

Thereafter, the dilation catheter 10 (with its balloon 14 or other dilator still in its non-expanded state) is advanced over the guidewire GW to a position where the balloon 14 or other dilator is positioned within the sinus ostium. The endoscope 60 may be used to view the advancement and positioning of the dilation catheter 10. Although the distal portion of the balloon 14 or other dilator will be within the sinus and out of the field of view of the endoscope 60, the endoscope 60 may be used to view the proximal end of the balloon 14 or other dilator and/or the optional marker 19 (if present) adjacent the proximal end of the balloon 14 or other dilator. Fluoroscopy may be used to image the radiographic markers 40, 42 and the ostium to confirm that the midregion 44 of the balloon 14 (or the appropriate portion of any other type of dilator) is positioned within the ostium.

After the balloon 14 or other dilator has been positioned within the ostium, the balloon 14 is inflated (or the other dilator is expanded) thereby dilating the ostium.

The balloon 14 is then deflated (or the dilator is returned to its non-expanded state), and the successful dilation of the ostium may be confirmed visually using the endoscope 60 and/or radiographically using a fluoroscope.

Thereafter, the dilation catheter 10, guidewire GW and guide catheter 70a-70f are removed.

Although the above described examples refer to use of a guide catheter 70a-70f and/or guidewire GW to guide the advancement of the dilation catheter 10 to its intended position within the ear, nose or throat, in some subjects and/or in some applications, the dilation catheter may be advanceable or maneuverable to its intended position without the use of a guide catheter 70a-70f and/or guidewire GW. For example, in some subjects, the dilation catheter 10 may be advanced into the sphenoid sinus ostium without the use of a guidewire GW or guide catheter 70a-70f. Alternatively, the flexible balloon portion may be manipulated with forceps to enable insertion in the ostium. Similar techniques may apply to access of the frontal and maxillary paranasal sinus ostia.

The fact that the system described herein includes a guide catheter 70a-70f that is separate from the dilation catheter 10 has certain advantages. For example, by having two separate devices, the operator has separate control of guide catheter placement and may, in some cases, elect not to actually advance the guide catheter 70a-70f into the ostium or (or frontal recess in the case of the frontal sinus). Rather, the operator may in some instances elect to maneuver the guide catheter 70a-70f to a position that is close to (e.g., aligned with) but not within the ostium or recess, and may then advance just the relatively flexible dilation catheter 10 into the ostium or recess. This may avoid damaging mucosal tissue and/or bone in the nasal cavity and/or of the ostium itself. Thus, the use of a guide catheter 70a-70f that is separate from the dilation catheter 10 allows flexibility of positioning and potentially less trauma than where a single rigid device (e.g., a rigid shafted dilation catheter) must be navigated to the desired location and then actually inserted into the ostium or other passageway to be dilated.

### Alternative Embodiment of a Balloon Dilation Catheter

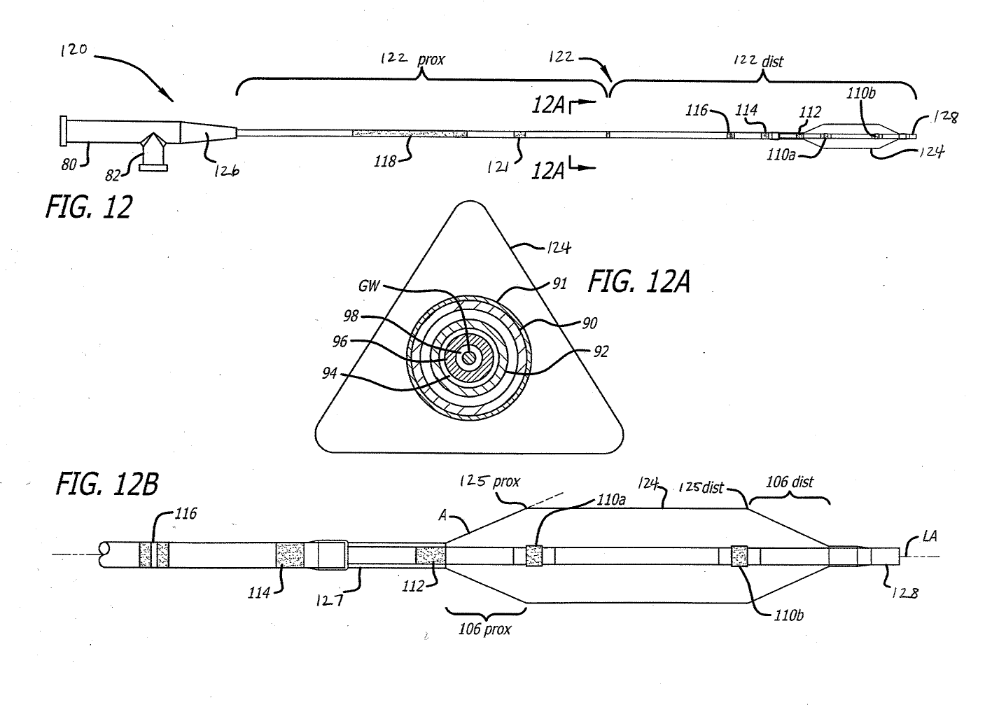

FIGS. 12, 12A and 12B show another example of a balloon dilation catheter device 120. In this embodiment, the dilation catheter device 120 includes an elongate catheter shaft 122 having a proximal shaft section 122prox that is substantially rigid and a distal shaft section 122dist that is more flexible than the proximal shaft section 122prox. An expandable dilator, such as a balloon 124, or other suitable mechanical or non-inflational dilator, is mounted on the distal shaft section 122dist, and a distal tip member 128 protrudes beyond the distal end of the balloon 124, as shown. Also, a proximal T hub 126 is attached to the proximal end of the proximal shaft section 122prox. This proximal T hub has a proximal Luer connector 80 and a side arm 82 having a female Luer connector that extends substantially perpendicular to the longitudinal axis of the hub 126, as shown. When compared to a typical Y hub, the side arm 82 of this T hub is further away from the proximal Luer connector 80 and is oriented at a right angle to the proximal Luer connector 80. Thus, tubing connected to this perpendicular side arm 82 is less likely to obscure or block the proximal Luer connector 80 than in a typical Y hub and the operator is less likely to confuse the proximal Luer connector 80 with the Luer connector on the side arm 82.

In various embodiments, the overall length of the catheter shaft 122 may be in the range of about 24 cm to about 30 cm and in one embodiment about 25 cm. The proximal shaft section 122prox may have a length in the range of about 9 cm to about 15 cm, and the distal shaft section 122dist may have a length in the range of about 5 cm to about 10 cm. In the embodiment shown in FIG. 12, the catheter shaft 122 has an effective length of 18.9 cm±0.3 cm, and an overall length of 20.0 cm±0.5 cm. Further, the proximal shaft section 122prox is 11.1 cm±0.2 cm in length and the distal shaft section 122dist has a flexible length of 7.75 cm±0.3 cm in length. The flexible length is measured from the proximal end of the distal shaft section to the distal shoulder 125dist of the balloon 124.

The “ineffective tip length” of the distal shaft section 122dist, from the distal shoulder 125dist of the balloon to the end of the distal tip member 128, is 1.1 cm±0.2 cm for a 7 mm balloon. For different balloon sizes, the ineffective tip length is 0.75 cm±0.2 cm for a 3.5 mm balloon, 0.9 cm±0.2 cm for a 5 mm balloon, and 1.0 cm+0.2 cm for a 6 mm balloon. Also, the distal tip member 128 is sufficiently flexible so that it is largely atraumatic (i.e., causes little or no damage to mucosal tissue upon contacting it during a procedure) and may have a radius shaped distal end.

Referring now to the cross sectional view of FIG. 12A, the proximal shaft section 122prox may include a rigid outer tube 90, a flexible middle tube 92 disposed substantially coaxially within the lumen of the rigid outer tube 90, and an inner tube 96 disposed substantially coaxially within the lumen of the middle tube 92. (A guidewire GW is shown coaxially within the lumen of the inner tube 96.) In this embodiment, the outer tube 90 is formed of stainless steel hypotube or support tube having an outer diameter of about 0.076 inches and an inner diameter of about 0.068 inches. The relatively larger outer diameter of outer tube 90 compared to the outer tube 30 of balloon catheter 10, helps decrease the inflation time of the balloon 124. As an alternative to stainless steel hypotube, this outer tube 90 may be formed of rigid non-metallic material such as polyetheretherketone (PEEK) or other rigid plastics suitable for such application. Alternatively, other rigid reinforcing members may be used in, or in lieu of, the outer tube, such as wires (round, flat, square or of other cross section), partial tubes (e.g., arcs), etc. Also, in this particular example, the middle tube 92 is formed of Pebax having an inner diameter of 0.055 inches, an outer diameter of 0.065+/−0.003 inches. The inner tube 96 is formed of polyether block copolymer tubing (e.g., Pebax® Resin, Arkema, Inc., Philadelphia, Pa.) having an inner diameter of at least 0.036 inches, and preferably having an inner diameter of 0.038 inches and an outer diameter of 0.048 inches. Having an inner tube 96 with an inner diameter of at least 0.036 inches allows the balloon catheter 120 to be compatible with multiple types of guidewires, including a lighted guidewire, such as the Acclarent Relieva Luma™ Sinus Illumination Guidewire, which has an outer diameter of 0.0354 inches.

The outer tube 90 terminates at the end of the proximal shaft section 122prox. The middle tube 92 and inner tube 96 extend beyond the distal end of the outer tube 90, forming the distal shaft section 122dist.

As seen in FIG. 12A, a polyether block copolymer film laminate 91 (e.g., Pebax® Resin, Arkema, Inc., Philadelphia, Pa.) covers the outer surface of the catheter shaft 122 from the proximal hub 126 to the balloon 124. Film laminate 91 may be applied to shaft 72 using known heat shrinking techniques. This laminate 91 provides a smooth outer surface and smoothes the step-down in diameter from the distal end of the proximal shaft section 122prox to the proximal end of the distal shaft section 122dist (i.e., it provides a smooth surface over the distal end of the outer tube 90 and the adjacent outer surface of the middle tube 92). The smooth step down may also be formed by an adhesive fillet. In other embodiments, the smooth step down may be formed by tapering or chamfering the structure of the distal end of the proximal shaft, eliminating the need for a laminate or adhesive.

The proximal end of the middle tube 92 extends into and is secured to the hub 126, distal to side arm Luer connector 82. The proximal end of the inner tube 96 extends into and is secured within hub 126, proximal to the side arm Luer connector 82 and in direct alignment and fluid communication with proximal Luer connector 80. The distal end of the middle tube 92 terminates within the balloon 124, and the proximal end of the dilator is secured to the outer surface of the middle tube. The distal end of the inner tube 96 also extends through the balloon 124 and protrudes distally beyond the balloon 124, forming the relatively flexible distal tip member 128 as shown in FIG. 12. The distal end of the balloon 124 is secured to the outer surface of the inner tube 96. In this manner, the inner tube lumen 98 extends through the entire catheter shaft 122 from the proximal Luer connector 80 through the distal tip 128 and may be used as a guidewire lumen or as a working lumen for infusion of irrigation solution, medicaments, contrast media or other substances and/or for aspiration of blood, fluids or debris. Guidewires that may be advantageously used in conjunction with this dilation catheter 120 may have a length of 60 cm to 80 cm and may be either 0.014 inch or 0.035 inch, such as those commercially available as the Relieva® Sinus Guidewires (Acclarent, Inc., Menlo Park, Calif.) or sizes in between such as 0.018 inch, 0.020 inch, or 0.033 inch. Although the drawings show an over-the-wire catheter 120 having a guidewire lumen that extends through the entire length of the catheter 120, guidewire lumens extending less than the entire length of the catheter (e.g., rapid exchange guidewire lumens) may be used as an alternative to the over-the-wire lumen shown. Additionally, in some embodiments, rather than advancing the catheter 120 over a guidewire, the catheter 120 may be equipped with a fixed guidewire tip such as any of those described in U.S. patent application Ser. No. 11/438,090 entitled Catheters with Non-Removable Guide Members Useable for Treatment of Sinusitis, the entire disclosure of which is expressly incorporated herein by reference.

The inner tube lumen 98 may be lined or coated with a lubricious material to facilitate passage of the guidewire GW through the lumen 98. The diameter of the inner tube 96 may be changed to accommodate guidewires of different diameter. In the particular embodiment described, the inner tube lumen 98 is sized to receive a 0.035 inch diameter guidewire GW. The inner tube lumen 98 may be internally lined or coated with a 2% solution of linear polydimethylsiloxane (PDMS) (e.g., Dow Corning® 360 Medical Fluid, Dow Corning Corporation, Midland, Mich.) diluted in isopropyl alcohol or another silicone material (such as a 2% solution of Dow-Corning MDX4-4159 in isopropyl alcohol). The coating is cured at room temperature.

The luminal space 94 between the outer surface of the inner tube 96 and the inner surface of the middle tube 92 is in fluidic communication with the side arm Luer connector 82 and extends to the interior of the balloon 124. Thus, this luminal space 94 serves as the passageway through which inflation fluid passes into and out of the balloon 124. The size of the luminal space 94 and the relatively short length of the catheter shaft 122 are optimized to minimize drag on inflation fluid passing through the luminal space 94 and allow for rapid deflation of the balloon 124. The clearance of 0.006 to 0.007 inches between the inner and outer members is desired for catheter length of 20-35 cm. The desired deflation time is less than or equal to about 5 seconds, and the deflation time is measured with application of negative pressure on the inflation/deflation lumen using a 20 cc inflation device that is filled with 10 cc contrast/saline mixture.

FIGS. 12A and 12B show details of the balloon 124. In this embodiment, the balloon 124 is a non-compliant balloon formed of polyethylene teraphthalate (PET) film having a thickness of 0.8 mils. As seen in FIG. 12A, the balloon 124 has a triangular or tri-fold (or approximately triangular) cross-sectional shape 104 in a partially inflated state. (“Cross-sectional,” when used in the present application to describe a shape of a balloon, refers to a cross section taken through the balloon 124 along a plane perpendicular to the longitudinal axis of the balloon 124 and within the working length of the balloon 124.) In alternative embodiments, the balloon 124 may have any suitable geometry in a partially inflated state, such as a round shape or any suitable non-round shape. The approximately triangular shape may facilitate wrapping and/or re-wrapping the balloon 124 around the outer tube 90. In use, the triangular cross-sectional shape of the balloon 124 allows the balloon 124, when deflated, to more easily re-wrap and pass back through the guide catheter for removal from a patient.

Referring to FIG. 12B, various embodiments of the balloon catheter 120 having variously sized balloons 124 may be provided, such as but not limited to the following sizes (diameter×effective length of the balloon in millimeters): 5×16, 6×16, 7×16, 5×24, 7×24, 3.5×12. Other balloon sizes may also be available. The working length (or “effective length”) of the balloon is measured from a proximal shoulder 125prox to a distal shoulder 125dist of the balloon 124. In some embodiments, a number of catheters 120 having a number of balloon sizes may be provided to a user, so that the physician user may choose one or more sizes based on the anatomy to be treated, physician preference and/or the like. In one embodiment, the balloon 124 may have a rated burst pressure of at least about 10 to about 16 atmospheres and preferably about 14 to about 16 atmospheres.

The balloon 124 also includes tapered proximal and distal end regions 106prox and 106dist. In some embodiments, each of the two tapered end regions 106prox, 106dist may have the same length. This length of the tapered regions 106prox, 106dist may be different for differently sized balloons 124. For example, in one set of balloon 124 embodiments, a balloon 124 having a diameter of at about 7 mm may have a taper length of about 6 mm, a balloon 124 having a diameter of at about 6 mm may have a taper length of about 5 mm, a balloon 124 having a diameter of at about 5 mm may have a taper length of about 4 mm, and a balloon 124 having a diameter of at about 3.5 mm may have a taper length of about 2.5 mm.

The tapered end regions 106prox, 106dist are tapered at angle A relative to the longitudinal axis LA of the catheter shaft 122 on which the balloon 124 is mounted. This angle of taper A may be in the range of about 10 degrees to about 30 degrees. In the particular example shown in the drawings, such angle of taper A is 20 degrees. This 20 degree angle of taper provides improved transition from balloon working length to the necks, lower profile, improved crossing, improved track, easier withdrawal in the sinus guide after balloon deflation. It also provides optimal performance with minimum increase of overall balloon length.

As best shown in FIG. 12B, the balloon 124 includes an extended balloon neck 127. In this embodiment, the balloon neck is about 1 cm in length. A proximal end of the balloon neck 127 may be bonded to the distal shaft portion 122dist. The extended balloon neck provides a separation between the bond to the shaft 122 and the tapered end region 106prox. This separation allows a marker to be disposed on the shaft and aligned with the proximal end of the balloon (at the proximal taper) without being disposed on or near the bond (adhesive) that secures the balloon to the shaft.

As shown in FIGS. 12 and 12B, in some embodiments, direct visualization markers and/or radiographic markers may be disposed along the catheter shaft 122. Generally, “direct visualization markers” refers to markers that may be viewed during use with the naked eye or by use of an endoscope, while radiographic markers include radiopaque material and are viewed using a radiographic device such as intra-operative fluoroscopy. In one embodiment, at the distal end, there is a first distal radiographic marker 110a, which has a proximal edge aligned with the location where the proximal taper 106prox meets the effective length of the balloon 124. There is also a second distal radiographic marker 110b, which has a distal edge aligned with the location where the distal taper 106distal meets the effective length of the balloon 124. The distance across the outside edges of the distal markers 110a and 110b is about 1.6 cm±0.2 cm and represents the effective length of the balloon 124. The distal markers 110a and 110b may be platinum marker bands. In this embodiment, the distal markers help to ensure that the balloon catheter 120 is in a straight position inside the guide during the device loading and preparation.

Direct visualization markers can be positioned in a number of locations along the catheter shaft 122. Although one embodiment is described here with reference to FIGS. 12 and 12B, other variations may be substituted in alternative embodiments. In one embodiment, shaft 122 may have a dark color, such as black, dark blue, dark grey or the like, and markers may have a light color, such as white, green, red or the like. In some embodiments, markers may have different colors and/or different widths to facilitate distinguishing the markers from one another during use. This contrast in colors may facilitate viewing the markers in a darkened operation room and/or when using an endoscope inside a patient in the presence of blood.

In one embodiment, there may be a first distal shaft marker 112 (or “endoscopic marker,” since it is typically viewed during use via an endoscope) disposed on the shaft 122 at a location such that its distal edge aligns with the location where the proximal taper of the balloon 124 meets the catheter shaft 122. The extended balloon neck 127 allows the first endoscopic marker 112 to be placed on the shaft and away from any adhesive bonding used to secure the proximal end of the balloon neck to the shaft. The first endoscopic marker 112 indicates to the user the ending location of the balloon 124 and indicates that the balloon has exited the guide during a procedure. In one embodiment, the first endoscopic marker 112 may be about 2 mm wide.

A second distal shaft marker 114 is disposed on the shaft 122 such that the distal edge of the marker is 1 cm±0.2 cm from the location where the proximal taper of the balloon 124 meets the catheter shaft 122. This marker indicates to the user that the shaft location is 1 cm away from the end of the balloon indicating that the balloon has extended from the guide during the procedure. In one embodiment, the second distal shaft marker may be about 2 mm wide and white in color, while the first marker is about 2 mm and green in color. Of course, any of a number of different size and color combinations may be used alternatively.

A third distal shaft marker 116 is disposed on the shaft 122 such that the distal edge of the marker is 1 cm±0.1 cm from the distal edge of the second distal shaft marker 114. As shown in FIG. 12B, the third distal shaft marker is a double marker to distinguish the second and third distal shaft markers 114 and 116 from one another. The third distal shaft marker 116 indicates the shaft location 2 cm away from the end proximal end of the balloon 124, thus indicating the distance the balloon has extended from the guide during the procedure. In one embodiment, the two markers forming the third distal shaft marker 116 are each 0.75 mm wide and white in color, however, the size and color of the marker can be changed in alternative embodiments. The differences in the first, second and third distal shaft markers' color, length and number of marks give the indication of the relative location proximal to the balloon under endoscopic visibility. Using an endoscope, the physician user can identify the length of catheter that has been advanced and retracted out of a guide catheter and/or can approximate a location of the balloon 124 relative to patient anatomy such as a paranasal sinus ostium, other paranasal sinus opening, or other openings in the ear, nose or throat. This approximation of balloon position may be very useful in circumstances when the balloon 124 has been advanced far enough into an anatomical location that the balloon 124 can no longer be viewed via endoscope. For example, using the three endoscopic markers, the user is able to endoscopically gauge the distance the catheter has advanced into the frontal recess once the proximal portion of the balloon is no longer visible. Of course, in alternative embodiments, distal shaft markers having different numbers, sizes, colors and positions along the catheter shaft may be used.

In some embodiments, in addition to one or more distal shaft markers, one or more proximal shaft markers may be disposed along the proximal portion of catheter shaft 122. In general, such proximal shaft markers may be viewed directly by a physician, without using an endoscope, to indicate to the physician a location of the balloon 124 of the catheter 120 relative to a guide catheter through which the balloon catheter 120 is being advanced. As with the distal shaft markers, the proximal shaft markers may have any suitable width, color, number, position and the like. In one embodiment, for example, as shown in FIG. 12, two proximal shaft markers 118, 121 may have a light color to contrast with a dark colored shaft 122 and increase visibility in a darkened operating room. The more proximal of the proximal markers 118 (or the “first proximal shaft marker”) may indicate that a tip of the balloon catheter 124 is at a distal end of the guide catheter and that the balloon 124 has exited the distal end of the guide catheter as the marker 118 passes into the proximal end of the guide catheter. The more distal of the proximal markers 121 (or the “second proximal shaft marker”) may indicate to a user that the balloon 124 is just proximal to a curve in a guide catheter when marker 121 is located at the proximal end of the guide catheter.

In one embodiment, the first proximal shaft marker 118 is disposed on the shaft 122 such that the length from the proximal end of the proximal balloon taper 106 to the proximal end of the first shaft marker is 13.1 cm±0.2 cm. The first proximal shaft marker is 4.1 cm±0.1 cm in length for a 7×24 mm balloon catheter. The length of the first proximal shaft marker 118 can vary depending on the size of the balloon catheter. The length of the first proximal shaft marker 118 may be determined by adding the length of the distal tip 128, the effective or working length of the balloon 124, and the lengths of the two balloon taper sections. Also, the first proximal shaft marker is preferably white in color, however, other light colors, such as grey, can be used as well.

The second proximal shaft marker 121 is disposed on the shaft 122 distally from the first proximal shaft marker 118. The second proximal shaft marker 121 is positioned such that the distal tip of the catheter 120 is 11.4 cm±0.2 cm from the distal edge of the second proximal shaft marker 121. Also, the second proximal shaft marker 121 has a length of 3 mm±2 mm. It is preferred that the second shaft proximal marker 121 is white in color, however, other light colors, such as grey, can be used as well.

When the balloon catheter 120 is inserted into a guide, a user may visualize the first and second proximal shaft markers 118 and 121 to determine the position of the distal tip and the balloon 124 of the balloon catheter 120 relative to the sinus guide catheter. For instance, when the second proximal shaft marker 121 is aligned with the proximal opening of the guide catheter, the user will know that the balloon 124 is proximal to the curve of the guide catheter. The position of the second proximal shaft marker 121 helps to visually ensure that the balloon catheter 120 is properly loaded into the sinus guide catheter. When the distal edge of the first proximal shaft marker 118 is aligned with the proximal opening of the guide catheter, the user knows that the distal tip of the balloon catheter 120 is beginning to exit the guide catheter, and when the proximal edge of the first proximal shaft marker is aligned with the proximal opening of the guide catheter, the user knows that the balloon is completely out of the guide catheter.

The visible markers 114, 116, 118 and 121 are preferably light in color, such as white as indicated above, to contrast with a dark color of the shaft 122, which is preferably black. The high contrast between these visible markers and the shaft helps view the markers in a low light environment. Also, the high contrast allows the user to view directly with an endoscope the markers and know where the balloon 124 is located relative to a sinus ostium. Furthermore, the color contrast is useful during the procedure when the field is full of blood and/or mucus to view the markers and know the position of the balloon. Of course, any other suitable contrasting color combination may be used. In one embodiment, for example, the catheter shaft 122 may be light colored, and the markers 114, 116, 118 and 121 may be dark colored.

The alternative embodiment of the balloon catheter 120 is used in a similar manner to the first embodiment of the balloon catheter 10 as described above. Further, separate features of the balloon catheters 10 and 120 may be incorporated into or used with either embodiment.

The invention has been described with reference to certain examples or embodiments of the invention, but various additions, deletions, alterations and modifications may be made to those examples and embodiments without departing from the intended spirit and scope of the invention. For example, any element or attribute of one embodiment or example may be incorporated into or used with another embodiment or example, unless otherwise specified or if to do so would render the embodiment or example unsuitable for its intended use. Also, where the steps of a method or process have been described or listed in a particular order, the order of such steps may be changed unless otherwise specified or unless doing so would render the method or process unworkable for its intended purpose. All reasonable additions, deletions, modifications and alterations are to be considered equivalents of the described examples and embodiments and are to be included within the scope of the following claims.

What is claimed:

1. A balloon dilation catheter device useable for dilating an opening in a paranasal sinus of a patient, the device comprising:
a catheter shaft having a longitudinal axis, an inflation lumen, a distal end, a proximal end, a proximal shaft section that is substantially rigid and a distal shaft section that is more flexible than the proximal shaft section, wherein the catheter shaft has a first color;
an inflatable balloon disposed on the distal shaft section in fluid communication with the inflation lumen, wherein the balloon has a non-circular cross-sectional shape, in a plane perpendicular to the longitudinal axis of the catheter shaft, when the balloon is partially inflated;
a first proximal shaft marker disposed on the proximal shaft section and having a second color that significantly contrasts with the first color of the catheter shaft, wherein the first proximal shaft marker allows a user to approximate a position of the balloon relative to a guide catheter through which the balloon catheter is advanced by visualizing a position of the first proximal shaft marker relative to a proximal end of the guide catheter; and
a first distal shaft marker disposed on the distal shaft section a selected distance proximal to a proximal end of the balloon and having a color selected from the group consisting of the second color and a third color, wherein the third color significantly contrasts with the first color of the catheter shaft.

2. The device of claim 1, further comprising a second proximal shaft marker disposed on the proximal shaft section distally from the first proximal shaft marker and having a color selected from the group consisting of the second color, the third color and a fourth color, wherein the fourth color significantly contrasts with the first color of the catheter shaft, and wherein the first shaft marker has a greater length than the second shaft marker.

3. The device of claim 2, wherein the length of the first proximal shaft marker is equal to a length from a proximal end of the inflatable balloon to the distal end of the catheter shaft, wherein the first proximal shaft marker is spaced from the distal end of the catheter shaft such that it allows the user to approximate when the distal end of the shaft is located at a distal end of the guide catheter and when the proximal end of the balloon exits the guide catheter, based on a location of the first proximal shaft marker relative to a proximal end of the guide catheter, and wherein the second proximal shaft marker allows the user to approximate when the distal end of the catheter shaft is located just proximal to a curve in the guide catheter, based on a location of the second proximal shaft marker relative to a proximal end of the guide catheter.

4. The device of claim 2, further comprising a second distal shaft marker disposed on the distal shaft section proximal to the first distal shaft marker and having a color selected from the group consisting of the second color, the third color, the fourth color and a fifth color wherein the fifth color significantly contrasts with the first color of the catheter shaft.

5. The device of claim 4, wherein the second distal shaft marker is disposed at a selected distance proximally from the first distal shaft marker, and wherein the first and second distal shaft markers have different appearances.

6. The device of claim 5, wherein the first distal shaft marker is disposed approximately one centimeter from the proximal end of the balloon and the second distal shaft marker is disposed approximately two centimeters from the proximal end of the balloon.

7. The device of claim 4, further comprising a third distal shaft marker disposed on the distal shaft section at the proximal end of the balloon.

8. The device of claim 1, further comprising:
a first radiopaque marker disposed on the distal shaft section within the balloon; and
a second radiopaque marker disposed on the distal shaft section distally from the first radiopaque marker within the balloon;
wherein the first and second radiopaque markers are disposed a distance apart from one another to indicate the effective length of the inflatable balloon.

9. The device of claim 1, wherein the cross-sectional shape of the balloon is approximately triangular.

10. The device of claim 1, wherein the inflatable balloon has a balloon neck extending from the balloon and proximally along the catheter shaft.

11. The device of claim 10, further comprising an endoscopic marker disposed on the distal shaft section and underneath the balloon neck.

12. A system for treating a disease or disorder of paranasal sinus of a patient, the system comprising:
a guide catheter insertable into a head of the subject, the guide catheter having a substantially rigid shaft, a proximal opening, a distal opening and a lumen extending between the proximal opening and the distal opening; and
a balloon catheter device, comprising:
a catheter shaft having a longitudinal axis, an inflation lumen, a distal end, a proximal end, a proximal shaft section that is substantially rigid and a distal shaft section that is more flexible than the proximal shaft section, wherein the catheter shaft has a first color;
an inflatable balloon disposed on the distal shaft section in fluid communication with the inflation lumen, wherein the balloon has a non-circular cross-sectional shape, in a plane perpendicular to the longitudinal axis of the catheter shaft, when the balloon is partially inflated;
a first proximal shaft marker disposed on the proximal shaft section and having a second color that significantly contrasts with the first color of the catheter shaft, wherein the first proximal shaft marker allows a user to approximate a position of the balloon relative to a guide catheter through which the balloon catheter is advanced by visualizing a position of the first proximal shaft marker relative to a proximal end of the guide catheter; and
a first distal shaft marker disposed on the distal shaft section a selected distance proximal to a proximal end of the balloon and having a color selected from the group consisting of the second color and a third color, wherein the third color significantly contrasts with the first color of the catheter shaft;
wherein the balloon catheter is sized to pass through the lumen of the guide catheter.

13. The system of claim 12, wherein the balloon catheter includes a guidewire lumen.

14. The system of claim 13, further comprising a guidewire that is advanceable through the guidewire lumen.

15. The system of claim 12, wherein the balloon catheter has an inflation fluid lumen through which inflation fluid passes into and out of the inflatable balloon and wherein the inflation fluid lumen is sized so that, after the inflatable balloon has been inflated to a working diameter, the inflatable balloon will deflate in less than 5 seconds with application of negative pressure to the inflation fluid lumen by a balloon catheter inflation and deflation device.

16. The system of claim 12, further comprising an irrigation catheter sized for advancement through the guide catheter into a paranasal sinus.

17. A method for dilating a paranasal sinus ostium of a paranasal sinus of a patient, the method comprising:
advancing a guide catheter into a head of a patient such that a distal end of the guide catheter is positioned within or near a paranasal sinus ostium of a paranasal sinus;
inserting an endoscope into the patient's head;
advancing a balloon catheter through a lumen of the guide catheter such that a balloon of the catheter passes out of the distal end of the guide catheter;
viewing, with the endoscope, a first distal shaft marker disposed on a shaft of the balloon catheter a first known distance from the balloon;
viewing, with the endoscope, a second distal shaft marker disposed on the shaft a second known distance from the balloon;
approximating a location of the balloon relative to the paranasal sinus ostium, using the first and second distal shaft markers and their known distances from the balloon; and
expanding the balloon of the balloon catheter to reshape or break bone underlying mucosa of the paranasal sinus ostium and dilate the ostium.

18. A method as in claim 17, further comprising viewing a first proximal shaft marker during the step of advancing the balloon catheter, wherein when a distal end of the first proximal shaft marker enters a proximal end of the guide catheter a distal end of the balloon catheter shaft is located approximately at the distal end of the guide catheter, and wherein when a proximal end of the distal shaft marker enters the proximal end of the guide catheter a proximal end of the balloon of the catheter is located approximately at the distal end of the guide catheter.

19. A method as in claim 18, further comprising viewing a second proximal shaft marker during the step of advancing the balloon catheter, wherein the second proximal shaft marker is disposed distal to the first proximal shaft marker, and wherein when the second proximal shaft marker is located approximately at the proximal end of the guide catheter, the distal end of the balloon catheter is located immediately proximal to a curve in the distal end of the guide catheter.

20. A method as in claim 17, wherein the first distal shaft marker is located proximal to the balloon and the second distal shaft marker is located distal to the balloon.

21. A method as in claim 20, wherein the first and second distal shaft markers are located proximal to the balloon.

22. A method as in claim 21, wherein the first distal shaft marker is located approximately one centimeter proximal to a proximal end of the balloon, and the second distal shaft marker is located approximately two centimeters proximal to the proximal end of the balloon.

23. A method as in claim 22, further comprising viewing a third distal shaft marker located at the proximal end of the balloon.

24. A method as in claim 17, further comprising advancing a guidewire through the guide and through the ostium before advancing the balloon catheter, wherein the balloon catheter is advanced over the guidewire and through the guide.

25. A method as in claim 17, further comprising:
removing the balloon catheter through the guide catheter;
advancing an irrigation catheter through the guide catheter into the paranasal sinus; and
irrigating the sinus using the irrigation catheter.

26. A device as in claim 4, wherein the first color of the catheter shaft is a dark color and the colors of the markers are light colors.

27. A device as in claim 26, wherein the first color is black and the colors of the markers are selected from the group consisting of white and green.

28. A device as in claim 5, wherein the first distal shaft marker comprises a single band, and the second distal shaft marker comprises a double band.
